SEQUENCE LISTING (3 sequences)

SEQ ID NO: 1 - protein, 183 aa, Homo sapiens
QEFLLRVEPQNPVLSAGGSLFVNCSTDCPSSEKIALETSLSKELVASGMGWAAFNLSNVTGNSRILCSVYCNGSQITGSSNITVYGLPERVELAPLPPWQPVGQNFTLRCQVEGGSPRTSLTVVLLRWEEELSRQPAVEEPAEVTATVLASRDDHGAPFSCRTELDMQPQGLGLFVNTSAPRQ

SEQ ID NO: 2 - protein, 181 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQ

SEQ ID NO: 3 - protein, 188 aa, Homo sapiens
KVFEVHVRPKKLAVEPKGSLEVNCSTTCNQPEVGGLETSLNKILLDEQAQWKHYLVSNISHDTVLQCHFTCSGKQESMNSNVSVYQPPRQVILTLQPTLVAVGKSFTIECRVPTVEPLDSLTLFLFRGNETLHYETFGKAAPAPQEATATFNSTADREDGHRNFSCLAVLDLMSRGGNIFHKHSAPKM

(19) United States
(12) Patent Application Publication
(10) Pub. No.: US 2013/0195940 A1
(43) Pub. Date: Aug. 1, 2013

(54) METHOD TO INHIBIT RECRUITMENT OF MONOCYTES AND MACROPHAGES BY AN ICAM-3 INHIBITOR

(71) Applicant: Andrew Devitt, Birmingham (GB)

(72) Inventor: Andrew Devitt, Birmingham (GB)

(73) Assignee: Aston University, Birmingham (GB)

(21) Appl. No.: 13/877,956

(22) PCT Filed: Oct. 5, 2011
(86) PCT No.: PCT/GB2011/001448

§ 371 Date: Apr. 5, 2013

(30) Foreign Application Priority Data
Oct. 6, 2010 (GB) 1016864.9

(51) Int. Cl. C07K 16/28; A61K 38/17; G01N 33/50

(52) U.S. Cl. CPC C07K 16/2821; G01N 33/5047; A61K 38/1774
USPC 424/400; 424/173.1; 424/153.1; 424/135.1; 424/133.1; 530/388.7; 435/7.1; 435/375; 514/20.9

(57) ABSTRACT

The present disclosure relates generally to methods and materials for modulating the recruitment of macrophages or monocytes to sites at which they may contribute to disease initiation or progression. Embodiments of the disclosure comprise providing a modulator of the activity of ICAM-3 or proximal to the site.

### TECHNICAL FIELD

The present invention relates generally to methods and materials for modulating the recruitment of macrophages or monocytes to sites at which they may contribute to disease initiation or progression.

### BACKGROUND ART

Monocytes and macrophages are phagocytic white blood cells which act in both non-specific and specific defense mechanisms in vertebrates. Their role is to phagocytose (engulf and then digest) dead and dying cells, cellular debris and pathogens either as stationary or as mobile cells, and to stimulate lymphocytes and other immune cells to respond to the pathogen. When attracted to sites of inflammation these cells can help to ‘close down’ the inflammation once the function of the inflammation has been performed (e.g. killing invading bacteria or repairing damaged tissue). The function of monocytes and macrophages includes the removal of dead or dying cells. For example unwanted cells are removed by an active death programme (apoptosis) that culminates in rapid, non-phlogistic removal of cell corpses by recruited and resident phagocytes (monocytes and most importantly macrophages: MØ) of the innate immune system¹⁻³.

Phagocytic removal of cells dying by apoptosis is a complex sequential process comprising attraction, recognition, tethering, signalling and ultimately phagocytosis and degradation of cell corpses. Relatively little is known of the molecular mechanisms underlying apoptotic cell surface changes which recruit phagocytes and mediate apoptotic cell phagocytosis⁴. However it is believed that MØ recognise ‘flags’ on the dying cell surface that are bound and allow the dying cell to be eaten and destroyed. A wide range of molecules acting as apoptotic cell-associated ligands, phagocyte-associated receptors or soluble bridging molecules have been implicated within this process. For example it has been proposed that in microparticles a chemokine called fractalkine is involved in the attraction of phagocytic removal of cells dying by apoptosis (see “Microenvironmental influences of apoptosis in vivo and in vitro” Gregory & Pound Apoptosis, September; 15 (9): 1029-1049).

Notwithstanding the important role macrophages and monocytes play in the body during apoptosis or inflammation, the recruitment of these cells is also believed to play a role in the morbidity and mortality of a broad spectrum of diseases, including autoimmune diseases, granulomatous diseases, allergic diseases, infectious diseases, osteoporosis and coronary artery disease (see e.g. EP1012187).

Furthermore it has been suggested that macrophages and monocytes may contribute to cancer growth and metastasis through the secretion of various mediators and growth factors, and the production of proteases. Thus tumor growth and progression have been linked to inflammation and the presence of tumor-associated macrophages (see e.g. Nature Reviews Cancer 4, 71-78 (January 2004) “Opinion: Tumour-educated macrophages promote tumour progression and metastasis” Jeffrey W. Pollard; also WO2008/058021). Non-malignant monocytes and macrophages may also play a role in cancer-associated cachexia. Cachexia is associated with a chronic, systemic inflammatory response and the elevation of acute phase proteins (see, e.g. Tisdale, 2001, Nutrition 17:438-442).

Rollins et al. (U.S. Pat. No. 5,459,128) generally disclose analogs of monocyte chemoattractant protein-1 (MCP-1) that inhibit the monocyte chemoattractant activity of endogenous MCP-1 which is believed to be necessary for the recruitment of monocytes and other inflammatory cell types.

Nevertheless new therapies are always needed for treating diseases such as those described above. Thus it can be seen that the provision of novel interventions which could modulate macrophage/monocyte recruitment to diseased sites in the body would provide a contribution to the art.

### BRIEF DISCLOSURE OF THE INVENTION

The present inventors have demonstrated that ICAM-3 is released from dying cells and can thereby recruit macrophages to the site of the cells. ICAM-3 is a heavily glycosylated, leukocyte-restricted Immunoglobulin (Ig) Super-Family member and is composed of five Ig domains.

This finding has been supported, by way of example only, by showing that not only does the release of microparticles from apoptotic cells attract macrophages, but importantly that microparticles lacking ICAM-3 demonstrate significantly reduced attraction of macrophages and that antibodies to ICAM-3 can block the attraction of macrophages.

These findings are consistent with the observation that ICAM-3 expression reduces early in apoptosis. It is believed that reduced ICAM-3 is a consequence of shedding of microparticles containing ICAM-3 that subsequently act as potent chemoattractants for MØ. Thus, taken together, these data confirm that ICAM-3 can play an important role both in the recruitment of macrophages to inflammatory sites and the resolution of inflammation. ICAM3 is thus a rational target for therapeutic intervention into diseases characterised by recruitment of macrophages and\or monocytes (MMs).

It was previously known that ICAM-3 was involved in removing dead cells in vitro and thereby through inference from the body. For example it has been previously been shown that ICAM-3 undergoes a change of function as cells die so that it acts as a molecular ‘flag’ to mediate corpse removal⁵. That process was known to be involved in resolution of inflammation.

ICAM-3 and its role as an adhesion molecule and binding partner of LFA-1 is described in WO92/22323. Pharmaceuticals alleged to treat diseases mediated by LFA-1\ICAM-3 interactions are discussed in WO01/27102.

However it was not known that ICAM3 was actually released from dead or dying cells, and thereby was capable of chemoattracting macrophages to the site of the cells. Nor was it known that this process could be modulated by agents which interfere with ICAM3 function.

Furthermore the findings of the inventors provide for novel methods for modulating, and in particular inhibiting, the recruitment of macrophages and monocytes to certain sites of inflammation where their presence is undesirable.

Without intending to be bound by any particular theory, blocking or interfering with the migration of MMs that are attracted to the tumor sites in the cancer patient or to the inflamed tissues in a patient with an inflammatory disease can be of clinical benefit. By blocking the recruitment of these cells in a cancer patient, there will be reduced numbers of inflammatory infiltrating cells, which may lead to a reduced tumor burden and reduced tumor-associated cachetic or other symptoms.

In a patient with an autoimmune or inflammatory disease, blocking the recruitment of MMs will lead to reduced numbers of inflammatory infiltrating cells in the affected area(s). The result could be reduced swelling, pain, and symptoms associated with the autoimmune or inflammatory disease. Specifically, a preferred target site is the atherosclerotic plaque. At this site there is a vicious cycle of MMs recruitment to low level inflammation. Those cells once recruited then die causing the recruitment of more MMs leading to plaque growth until it ultimately becomes unstable leading to potential adverse consequences. Clearly inhibition of MMs recruitment in this context can be of clinical benefit.

### DETAILED DISCLOSURE OF THE INVENTION

Thus in one aspect of the invention there is provided a method for modulating the recruitment of macrophages and\or monocytes (MMs) to a site of cell injury or cell death, which method comprises providing a modulator of the activity of ICAM-3 in the proximity of the site.

For example the invention provides a method of inhibiting MMs chemoattractant activity by administering to a patient in need of treatment thereof an effective amount of the modulator.

In a less preferred embodiment, MØ could (for example) be attracted to a site of non-resolving inflammation by supplying ICAM-3 to the vicinity of the site, with the intention of increasing phagocytosis, for example in autoimmune disease.

However in preferred embodiments the modulator is an inhibitor, which can be used for example for functional blockade of ICAM-3 around the site.

Cell death in this context means that the cell has suffered an irreversible loss of integrated cellular function, for example through necrosis or apoptosis.

For example necrosis will typically affect a contiguous group of cells, and will lead to cell swelling and lysis and loss of membrane integrity. Typically it will precipitate an inflammatory response. Necrosis may, by way of non-limiting example, be “secondary” in that results from failed clearance of apoptotic cells which then become necrotic

By contrast apoptosis may or may not lead to an inflammatory response, but is nevertheless typically associated with rapid phagocytosis.

Generally speaking, the site of cell injury or death will be one wherein the presence of said MMs is undesirable in, for example because its exacerbates or promotes an inflammatory cycle or gives rise to some undesirable symptom or outcome. Examples of such instances are described herein. In certain preferred embodiments the site is one of cell death not associated with inflammation.

As described below, the inhibitor in question will have the aim of inhibition (e.g. functional blockade) of the chemoattractant activity of ICAM-3 in respect of MMs. Preferably it will be selective for ICAM-3.

In an embodiment, administration of the ICAM-3 inhibitor decreases the number of MMs in vicinity of the site, or inhibits the migration of MMs to the site.

In another aspect there is provided methods of treating diseases associated with undesirable infiltrates that include monocytes and macrophages.

In aspects of the invention described herein it will be understood that the chemoattractant activity of ICAM-3 will generally be manifested as concentration gradient of ICAM-3, being highest at the site of the inured or dying cell or cells, The modulators of the invention may thus have the effect of inhibiting the chemoattractive effect of this gradient.

A method of treating a mammal with cancer or inflammatory disease with an inhibitor of the activity of ICAM-3 in the proximity of the site.

A method for reducing inflammation and tissue damage associated with autoimmune or inflammatory diseases in a mammal using an inhibitor of the activity of ICAM-3.

A method of treating cancer to delay progression, reduce tumor burden and/or reduce cancer-associated cachexia in a mammal in need thereof by administering to the patient an effective regimen of an ICMA-3 binding agent in the vicinity of the tumor.

The invention further provides inhibitors of ICAM-3 for use in these methods or for treating the diseases described herein, and use of such inhibitors in the preparation of pharmaceutical compositions for treating the diseases described herein. For example the invention provides pharmaceutical compositions for treating a mammal with cancer or autoimmune or inflammatory disease, and/or for preventing or delaying recurrence of such diseases in a patient.

Preferably the mammal is a human patient.

In some embodiments, the methods further include monitoring the number of MMs in the mammal e.g. at the site of cell death (e.g. plaque or tumor). The dosage of therapeutic agents can be adjusted based on the monitoring.

### ICAM-3

The agents and inhibitors used in the invention have the effect of modulating the chemoattractant activity of ICAM-3 in respect of MMs.

ICAM-3 is an adhesion molecule that was previously known to be involved in many cell-cell interactions. To the extent that it was believed to be involved in the emigration of blood cells out of the blood and to sites of inflammation, it was previously believed that such emigration was essentially mediated by the ICAM-3 present on the viable cells that migrated to the inflammation.

Generally speaking, the methods of the present invention generally do not seek to target ICAM-3 on the infiltrating phagocytic cells and its interaction with integrins in the well-established mode of action for ICAM-3 in extravasation of blood cells, but rather to block the ICAM-3 being released from dead and dying cells at the site of inflammation, or to block its effect once released.

As described in the Examples below, it is believed that ICAM-3 is released from dead and dying cells and subsequently acts as a potent chemoattractants for MO.

Without wishing to be bound by theory, ICAM-3 may be released from such cells as a result of the dynamic changes in the membranes of the cells e.g. as a result of zeiosis, ‘blebbing’, fragmentation into apoptotic bodies and release of microparticles, as well as release as an isolated factor.

Thus it will be understood that where the chemoattractant properties of ICAM-3 are referred to herein, the ICAM-3 will be non cell-associated, but may be in membrane-encapsulated or membrane-associated forms e.g. at the surface of the apoptotic body, bleb or microparticle. The Examples below used microparticles by way of demonstration of the invention but it will be appreciated that where this term is used, the invention applies correspondingly to “blebs” and so on.

### Preferred Inhibitors

Inhibitors of the activity of ICAM-3 in the context of the present invention are those which inhibit the interaction between ICAM-3 and MMs, and more preferably the chemoattractant activity demonstrated by ICAM-3 toward MMs.

Agents capable of decreasing the biological activity of ICAM-3 towards MMs may achieve their effect by a number of means. For instance, such agents may:

(i) Bind directly to ICAM-3 thereby reducing interaction between ICAM-3 and MMs;
(ii) Bind directly to relevant ICAM-3 receptors on MMs thereby reducing interaction between ICAM-3 and MMs;
(iii) Compete with ICAM-3 for MM binding, for instance by way of being structural analogs lacking the relevant biological activity of ICAM-3.

Such agents may be provided as described hereinafter, and then screened to confirm activity as described below.

Thus, according to one aspect of the present invention there is provided a method of providing\screening a compound to test whether or not the compound has efficacy for treating an indication described herein, comprising:

(i) providing an ICAM-3-derived agent;
(ii) determining its ability to inhibit chemoattraction between an chemoattractant which comprises ICAM-3 and a macrophage or monocyte.

In one embodiment analogs or mimetics of all or part of the ICAM-3 molecule may be utilised as agents of the invention. Preferred fragments, analogs or mimetics may be based on the sequence of Domain 1 and\or Domain 2 of ICAM-3 (see FIG. 9).

A preferred agent for use according to the present invention is an antibody molecule raised to ICAM-3 or a portion thereof, for example a monovalent antibody. By way of Example and support only, the present inventors provide an example antibody (termed ‘MA4’) which binds to a site on ICAM-3 that interferes with its interaction with MMs.

### Diseases to be Treated

Antibody molecules and other agents according to the invention may be used in a method of treatment or diagnosis of the human or animal body, such as a method of treatment (which may include prophylactic treatment) of such diseases or disorders. The methods may comprise administering to said patient an effective amount of the agent.

Thus the invention may be used in methods of treating pathologies associated with cell death in which MMs infiltration to the site of cell injury or death is undesirable, and where the dying cells bear ICAM-3. The invention may be applied to any such disease listed in the section describing diseases susceptible to treatment by the present invention hereinafter.

Preferably the invention is applied for treating diseases in which MMs infiltration to a site of cell injury or death is undesirable e.g. because it exacerbates or promotes an inflammatory cycle or gives rise to some undesirable symptom or outcome. For example a preferred treatment is in respect of non-resolving inflammatory sites.

The treatments may have as their purpose reduction of inflammation, pain and\or tissue destruction in inflamed tissues.

Examples of indications relevant to the present invention, and to which the invention is preferably applied, include:

### i) Atherosclerosis, and Other Forms of Local or Systemic Vasculitis

Diseases such as myocardial infarction, stroke and acute ischemia which are secondary to atherosclerosis;
Also emboli released from ruptured plaques and consequences of this (e.g. pulmonary embolus)
hypertension;
reperfusion injury;
aortic aneurysms;
vein graft hyperplasia;
angiogenesis;
hypercholesterolemia;
congestive heart failure;
Kawasaki's disease;
stenosis or restenosis, particularly in patients undergoing angioplasty;
rheumatoid arthritis, and glomerulonephritis

A preferred target site is the atherosclerotic plaque. Such plaques are generally believed to begin with a streak of fat in the arterial wall. This becomes a focus for inflammation.

Briefly, inflammatory cells, including those carrying ICAM-3, respond to chemotactic cues enter the affected site and then die by apoptosis. Other cells infiltrate to deal with the inflammation and death leading to a vicious cycle of recruitment, ‘eating’, death, more recruitment. Eventually the plaque may become unstable and rupture with devastating cardiac consequences.

### ii) Cancers

Preferred targets are cancers of leukocyte (i.e. ICAM-3-expressing) cell origin where there are tumour-associated macrophages.

Examples include Lymphomas such as Hodgkin's and Non-Hodgkin lymphoma (see Tables 1 and 2).

TABLE 1
Histopathologic Subtypes of Hodgkin Lymphoma (WHO Classification)

| Histologic | Morphologic | Tumor Cell | |
|---|---|---|---|
| Type | Appearance | Immunophenotype | Incidence |
| Classic | | | |
| Nodular | Dense fibrous | CD15+, CD30+, CD20− | 67% |
| sclerosis | tissue* surrounding | | |
| | nodules of Hodgkin | | |
| | tissue | | |
| Mixed | A moderate number | CD15+, CD30+, CD20− | 25% |
| cellularity | of Reed-Sternberg | | |
| | cells with a mixed | | |
| | background | | |
| | infiltrate | | |
| Lymphocyte- | Few Reed- | CD15+, CD30+, CD20− | 3% |
| rich | Sternberg cells | | |
| | Many B cells | | |
| | Fine sclerosis | | |
| Lymphocyte- | Numerous Reed- | CD15+, CD30+, CD20− | Rare |
| depleted | Sternberg cells | | |
| | Extensive fibrosis | | |
| Nodular | Few neoplastic cells | CD15−, CD30−, CD20+, | 3% |
| lymphocyte- | (lymphocytic or | EMA+ | |
| predominant | histiocytic cells or | | |
| | both) | | |
| | Many small B cells | | |
| | Nodular pattern | | |

TABLE 2
Subtypes of Non-Hodgkin Lymphoma (WHO Classification)

| Cell Origin | Tumor |
|---|---|
| Precursor B-cell | Precursor B-lymphoblastic leukemia/lymphoma* |
| tumor | |
| Mature B-cell | B-cell chronic lymphocytic leukemia/small |
| tumors | lymphocytic lymphoma† |
| | B-cell prolymphocytic leukemia† |
| | Lymphoplasmacytic lymphoma† |
| | Splenic marginal zone B-cell lymphoma (±villous |
| | lymphocytes)† |
| | Hairy cell leukemia† |
| | Plasma cell myeloma/plasmacytoma† |
| | Extranodal marginal zone B-cell lymphoma of the |
| | MALT type† |
| | Nodal marginal zone B-cell lymphoma (±monocytoid |
| | B cells)† |
| | Follicular lymphoma† |
| | Mantle cell lymphoma‡ |
| | Diffuse large B-cell lymphomas* (including |
| | mediastinal large B-cell lymphoma and |
| | primary effusion lymphoma) |
| | Burkitt's lymphoma* |
| Precursor T-cell | Precursor T-lymphoblastic lymphoma/leukemia* |
| tumor | |
| Mature T-cell | T-cell prolymphocytic leukemia† |
| tumors | T-cell granular lymphocytic leukemia* |
| | Aggressive NK cell leukemia* |
| | Adult T-cell lymphoma/leukemia* (HTLV 1-positive) |
| | Extranodal NK/T-cell lymphoma, nasal type* |
| | Enteropathy-type T-cell lymphoma* |
| | Hepatosplenic γ-δ T-cell lymphoma* |
| | Subcutaneous panniculitis-like T-cell lymphoma* |
| | Mycosis fungoides/Sezary syndrome† |
| | Anaplastic large cell lymphoma, T/null cell, primary |
| | cutaneous type* |
| | Anaplastic large cell lymphoma, T-/null-cell, |
| | primary systemic type* |
| | Peripheral T-cell lymphoma, not otherwise |
| | characterized* |
| | Angioimmunoblastic T-cell lymphoma* |

*Aggressive.
†Indolent.
‡Indolent but more rapidly progressive.
HTLV = human T-cell leukemia virus 1;
MALT = mucosa-associated lymphoid tissue;
NK = natural killer;
±= with or without.

Other preferred target cancers may be ovarian and breast carcinomas where it is understood attraction of MØ may drive tumorigenesis.

### 3. Granuloma-Associated Disease

Granulomas are seen in a wide variety of diseases, both infectious and non-infectious. Infections that are characterized by granulomas include tuberculosis, leprosy, histoplasmosis, cryptococcosis, coccidioidomycosis, blastomycosis and cat scratch disease. Examples of non-infectious granulomatous diseases are sarcoidosis, Crohn's disease, berylliosis, Wegener's granulomatosis, Churg-Strauss syndrome, pulmonary rheumatoid nodules and aspiration of food and other particulate material into the lung. Preferred granulomas is are those which are necrotic or caseous.

### Localised Delivery of Therapeutics

In the context of the present invention, it will be desirable to deliver the therapeutic agents locally to the site of cell death.

Localised administration to the area in need of treatment, as appropriate for the drug or agent, can be achieved, for example, and not by way of limitation, by local infusion during surgery; topical application, e.g. in conjunction with a wound dressing after surgery; by injection; by means of a catheter (such as an infusion or indwelling catheter, e.g., a needle infusion catheter); by means of a suppository; or by means of an implant, the implant being of a porous, non-porous, or gelatinous material, including membranes, such as silastic membranes, or fibers. In one embodiment, administration can be by direct injection at the site (or former site) of a cancer, tumor or neoplastic or pre-neoplastic tissue.

One preferred mode of administration employs precoating of, or otherwise incorporation into, indwelling devices, for which the optimal amount of agent (for example, antibody will be determined by means of appropriate experiments. Such embodiments are preferred where a high localised concentration is required.

The device coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices e.g., stents, catheters, peritoneal dialysis tubing, and the like.

A preferred means for delivering agents capable of modulating ICAM-3 activity to an atherosclerotic plaque is an impregnated stent inserted to open up the vessel occluded by the plaque.

More generally agents of the present invention may be targeted to a specific therapeutic site by linking the therapeutic agent to a moiety that specifically binds to a cellular component, e.g., antibodies or fragments thereof, lectins, and small molecule drugs, so as to form a therapeutic conjugate. Targeting of the therapeutic agents of the invention can result in increased concentration of the therapeutic agent at a specific anatomic location. Moreover, the linking of a therapeutic agent of the invention to a binding moiety may increase the stability of the therapeutic agent in vivo.

### Provision of Inhibitors

Agents of particular use in the present invention are those which inhibit the ability of ICAM-3 to bind, and hence chemoattract, MMs.

Agents capable of decreasing the biological activity of ICAM-3 towards MMs may achieve their effect by a number of means. For instance, such agents may:

(i) Bind directly to ICAM-3 thereby reducing interaction between ICAM-3 and MMs;
(ii) Bind directly to relevant ICAM-3 receptors on MMs thereby reducing interaction between ICAM-3 and MMs;
(iii) Compete with ICAM-3 for MM binding, for instance by way of being structural analogs lacking the relevant biological activity of ICAM-3.

As described below, in the light of the present disclosure suitable inhibitors can be provided by those skilled in the art. Inhibitors will typically be ‘small molecules’ or may be peptide agents or proteins.

Preferred agents are those directly interact with ICAM-3, for example Domains 1 and\or 2 of ICAM-3.

A preferred agent for use according to the present invention is an antibody molecule raised to ICAM-3 or a portion thereof, for example a monovalent antibody. These are discussed in more detail below.

As described herein, knowledge of the novel chemoattractant interaction between MMs and ICAM-3 provides for the use of ICAM-3 derived agents (e.g. antibody molecule or mimetic based on the sequence of Domains 1 and\or 2) to modify the recruitment of MMs to sites of dead or dying cells.

Such agents may be provided as described hereinafter, and then screened to confirm activity as described below.

Thus, according to one aspect of the present invention there is provided a method of providing\screening a compound to test whether or not the compound has efficacy for treating an indication described herein, comprising:

(i) providing an ICAM-3 derived agent;
(ii) determining its ability to inhibit chemoattraction between an ICAM-3 including attractant and a macrophage or monocyte.

This determination may be performed using varying amounts of the agent.

The ICAM-3 including attractant may be part of the membrane of an apoptotic cell.

This determination may be compared with a control which lacks ICAM-3, which may be part of the membrane of an ICAM-3 negative apoptotic cell.

The determination may be performed in a chemotaxis chamber, for example as described in the Examples hereinafter.

More generally, to determine whether the agent inhibits macrophage recruitment, varying amounts of the agent are mixed with cells in the presence of the chemoattractant. For example, a range of known concentrations of an agent is incubated with a defined number (e.g., 10⁴-10⁶) of human THP cells in individual wells of the top compartment of a trans-well plate. The ICAM-3 including chemoattractant at a concentration known to cause significant migration of THP-1 cells in the trans-well migration assay, is placed in the lower compartment. Cells are then incubated at 37° C. for a period sufficient to allow migration, e.g. 4 hours. After incubation, the cells are gently removed from the top of the filter with a pipette, 20 mM EDTA in simple PBS is added into each top well, and incubated for 20 minutes at 4° C. The filter is carefully flushed with media using a gentle flow, and removed. A standard curve consisting of a two-fold dilution series of THP-1 cells is prepared to accurately quantify the number of cells that have migrated. Migrated cells are stained with MIT stock dye solution which is added directly into each well (5 mg/ml in RPMI-1640 without phenol red, Sigma Chemical Co.) and incubated at 37° C. for 4 hours. The media is carefully aspirated from each well, and the converted dye is solubilized by DMSO. Absorbance of converted dye is measured at a wavelength of 595 nm using an ELISA plate reader. The number of migrated cells in each well is then determined by interpolation of the standard curve (see also Imai et al., J. Biol. Chem., 272, 15036 (1997)).

To assess whether the agent is cytotoxic, the same concentrations of agent are incubated with THP-1 cells. Agents which 1) are not cytotoxic at levels which inhibit migration, 2) are ineffective at inhibiting the negative control-induced migration, and 3) reduce or inhibit ICAM-3 induced THP-1 migration, are agents which fall within the scope of the invention.

To further determine whether a particular agent is useful in the practice of the methods of the invention, an animal model is identified for a human disease. Transgenic animal models for human disease may also be employed to identify agents useful in the methods of the invention.

For example, models of ICAM-3 induced macrophage recruitment associated with human disease include, but are not limited to, mice with xenografts of ICAM-3 expressing leukocyte tumour cells (and ICAM-3-deficient counterparts where appropriate) where a tumour may be established and infiltration of mouse macrophages may be assessed and modulated through administration of modulating agents.

The efficacy of an agent of the invention may be assessed by measuring the extent of inflammation, or the extent of macrophage infiltration of affected tissues. Macrophage infiltration can be detected by staining tissue sections with antibodies which specifically detect macrophages (e.g., mac-1 antiserum). Inflammation or other symptoms of disease may be detected by measuring appropriate clinical parameters, using techniques which are well known to those skilled in the art (e.g. measuring the reduction in the extent of vascular lipid lesion formation by histochemistry using oil red staining in accordance with Paigen, Arteriosclerosis, 10, 316 (1990)).

In one embodiment analogs or mimetics of all or part of the ICAM-3 molecule may be utilised as agents of the invention.

Such compounds may be based on the glycosylation of ICAM-3. However preferred fragments, analogs or mimetics are based on the sequence of Domain 1 and\or Domain 2 of ICAM-3 (see FIG. 9).

Preferred analogs or mimetics are those which compete with ICAM-3 in respect of their interaction with MMs but which do not exert the chemoattractant effect because they lack a domain or other structural feature capable of activating an intracellular signalling pathway in MMs.

Accordingly such agents are effective for reducing ICAM-3 mediated recruitment of MMs to sites of pathology.

Derivatives of peptide agents used according to the invention include derivatives that increase the half-life of the agent in vivo. Examples of derivatives capable of increasing the half-life of polypeptides according to the invention include peptoid derivatives, D-amino acid derivatives and peptide-peptoid hybrids.

Proteins and peptide agents according to the present invention may be subject to degradation by a number of means (such as protease activity at a target site). Such degradation may limit their bioavailability and hence therapeutic utility. There are a number of well-established techniques by which peptide derivatives that have enhanced stability in biological contexts can be designed and produced. Such peptide derivatives may have improved bioavailability as a result of increased resistance to protease-mediated degradation. Preferably, a derivative suitable for use according to the invention is more protease-resistant than the protein or peptide from which it is derived. Protease-resistance of a peptide derivative and the protein or peptide from which it is derived may be evaluated by means of well-known protein degradation assays. The relative values of protease resistance for the peptide derivative and peptide may then be compared.

Commercially available software may be used to develop peptoid derivatives according to well-established protocols.

Retropeptoids, (in which all amino acids are replaced by peptoid residues in reversed order) are also able to mimic proteins or peptides according to the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

A further embodiment of a modified form of peptides or proteins according to the invention comprises D-amino acid forms. In this case, the order of the amino acid residues is reversed. The preparation of peptides using D-amino acids rather than L-amino acids greatly decreases any unwanted breakdown of such derivative by normal metabolic processes, decreasing the amounts of the derivative which needs to be administered, along with the frequency of its administration.

As noted above, a preferred agent for use according to the present invention is an antibody molecule raised to ICAM-3 or a portion thereof, for example a monovalent antibody.

As antibodies can be modified in a number of ways, the term “antibody molecule” should be construed as covering any antibody molecule or substance having an antibody antigen-binding domain with the required specificity. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Preferred antibody molecules are monoclonal antibodies such as MA4 according to the invention including any functionally equivalent antibodies thereto and functional parts thereof. Examples of such equivalents and parts are described in more detail hereinafter.

“Specifically” in the context of the invention this means the ability to bind to ICAM-3 and thereby inhibit its chemoattractant interaction with MMs.

Typically, binding may be determined by means of a binding assay such as ELISA employing a panel of antigens, wherein it can be demonstrated that an antibody molecule according to the present invention will specifically recognise ICAM-3 but not other test antigens. As an alternative, a sensor such as a Biacore sensor may be used to compare or quantify binding.

The ability to inhibit the interaction of ICAM-3 with MMs in the presence of antibody may be tested as described above.

In one aspect the invention provides an antibody molecule which binds an epitope on ICAM-3 (e.g. in Domain 1 and\or 2) and which exhibits the desired inhibitory effect.

Thus a further aspect of the present invention provides an antibody molecule comprising a human antibody antigen-binding site which competes with MA4 for binding to ICAM-3.

In the light of the disclosure herein, antibodies specific for ICAM-3 and which may compete with MA4 for binding to the same or nearby ICAM-3 epitope can be readily provided. For example, a method may include bringing into contact a library of antibody molecules and said epitope, and selecting one or more specific antibody molecules of the library able to bind said epitope.

The library may be displayed on the surface of bacteriophage particles, each particle containing nucleic acid encoding the antibody VH variable domain displayed on its surface, and optionally also a displayed VL domain if present.

Following selection of specific antibody molecules able to bind the epitope and displayed on bacteriophage particles, nucleic acid may be taken from a bacteriophage particle displaying a said selected specific antibody molecule. Such nucleic acid may be used in subsequent production of a specific antibody molecule or an antibody VH variable domain (optionally an antibody VL variable domain) by expression from nucleic acid with the sequence of nucleic acid taken from a bacteriophage particle displaying a said selected specific antibody molecule.

Ability to specifically bind ICAM-3 may be further tested, also ability to compete with MA4 for binding to ICAM-3. An antibody molecule according to the present invention may bind ICAM-3 with the affinity of MA4.

Competition between antibody molecules may be assayed easily in vitro, for example by tagging a reporter molecule to one antibody molecule which can be detected in the presence of other untagged antibody molecule(s), to enable identification of antibody molecules which bind the same epitope or an overlapping epitope. Competition may be determined for example using ELISA or flow cytometry.

In testing for competition a peptide fragment of ICAM-3 may be employed, especially a peptide including the epitope of interest. A peptide having the epitope sequence plus one or more amino acids at either end may be used. Such a peptide may be said to “consist essentially” of the specified sequence. Antibody molecules according to the present invention may be such that their binding for ICAM-3 is inhibited by a peptide with or including the sequence given. In testing for this, a peptide with either sequence plus one or more amino acids may be used.

As noted above, preferred antibody molecules are monoclonal antibodies such as MA4 according or functionally equivalent antibodies or functional parts thereof.

In a preferred embodiment, the antibody molecule comprises the MA4 VH domain and/or the MA4 VL domain.

Generally, a VH domain is paired with a VL domain to provide an antibody antigen binding site, although as discussed further below a VH domain alone may be used to bind antigen.

In one preferred embodiment, the MA4 VH domain is paired with the MA4 VL domain, so that an antibody antigen binding site is formed comprising both the MA4 VH and VL domains. In other embodiments, the MA4 VH is paired with a VL domain other than the MA4 VL. Light-chain promiscuity is well established in the art.

One or more CDR's may be taken from the MA4 VH or VL domain and incorporated into a suitable framework.

Variants of the VH and VL domains of which the sequences are set out herein and which can be employed in antibody molecules for ICAM-3 can be obtained by means of methods of sequence alteration or mutation and screening. Such methods are also provided by the present invention.

Variable domain amino acid sequence variants of any of the VH and VL domains discussed herein may be employed in accordance with the present invention. Particular variants may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1. Alterations may be made in one or more framework regions and/or one or more CDR's.

Preferred substitutions are conservative substitutions.

Thus one aspect of the invention provides a method for obtaining an antibody antigen-binding domain specific for a desired ICAM-3 epitope, the method comprising providing by way of addition, deletion, substitution or insertion of one or more amino acids in the amino acid sequence of a VH domain of MA4 which is an amino acid sequence variant of the VH domain, optionally combining the VH domain thus provided with one or more VL domains, and testing the VH domain or VHNL combination or combinations to identify an antibody molecule or an antibody antigen binding domain specific for ICAM-3. Said VL domain may have an amino acid sequence which is substantially as set out herein.

An analogous method may be employed in which one or more sequence variants of a VL domain disclosed herein are combined with one or more VH domains.

A further aspect of the invention provides an antibody molecule such as a monoclonal antibody including any functionally equivalent antibody or functional parts thereof according to the present invention and as described herein wherein said antibody comprises a VL or VH domain as described above.

A further aspect of the invention provides a method of preparing an antibody molecule specific for ICAM-3, which method comprises:

(a) providing a starting repertoire of nucleic acids encoding a VH domain which either include a CDR3 to be replaced or lack a CDR3 encoding region;
(b) combining said repertoire with a donor nucleic acid encoding an amino acid sequence substantially as set out herein for a VH CDR3 such that said donor nucleic acid is inserted into the CDR3 region in the repertoire, so as to provide a product repertoire of nucleic acids encoding a VH domain;
(c) expressing the nucleic acids of said product repertoire;
(d) selecting an antibody molecule specific for ICAM-3; and
(e) recovering said specific antibody molecule or nucleic acid encoding it.

Again, an analogous method may be employed in which a VL CDR3 of the invention is combined with a repertoire of nucleic acids encoding a VL domain which either include a CDR3 to be replaced or lack a CDR3 encoding region.

Similarly, one or more, or all three CDRs may be grafted into a repertoire of VH or VL domains which are then screened for antibody molecules specific for ICAM-3.

A substantial portion of an immunoglobulin variable domain will comprise at least the three CDR regions, together with their intervening framework regions. Preferably, the portion will also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of specific antibody molecules of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including immunoglobulin heavy chains, other variable domains (for example in the production of diabodies) or protein labels as discussed in more details below.

Antibody molecules of the present invention include antibody molecules and other immunoglobulins whether natural or partly or wholly synthetically produced. The term covers any polypeptide or protein comprising an antibody binding domain. Specifically includes are antibody fragments which comprise an antigen binding domain are such as Fab, scFv, Fv, dAb, Fd; and diabodies. These things are discussed in more detail below.

Although in a preferred aspect of the invention specific antibody molecules comprising a pair of VH and VL domains are preferred, single binding domains based on either VH or VL domain sequences form further aspects of the invention. It is known that single immunoglobulin domains, especially VH domains, are capable of binding target antigens in a specific manner.

Thus in other aspects of the invention an antibody VH variable domain with the amino acid sequence of an antibody VH variable domain of an antibody molecule of the invention may be provided in isolated form, as may an antibody molecule comprising such a VH domain.

In the case of either of the single chain binding domains, these domains may also be used to screen for complementary domains capable of forming a two-domain antibody molecule able to bind ICAM-3.

This may be achieved by phage display screening methods using the so-called hierarchical dual combinatorial approach as disclosed in WO92/01047 in which an individual colony containing either an H or L chain clone is used to infect a complete library of clones encoding the other chain (L or H) and the resulting two-chain antibody molecule is selected in accordance with phage display techniques such as those described in that reference.

Antibody molecules of the present invention may further comprise antibody constant regions or parts thereof. For example, a VL domain may be attached at its C-terminal end to antibody light chain constant domains including human Cκ or Cλ chains, preferably Cκ chains. Similarly, an antibody molecule based on a VH domain may be attached at its C-terminal end to all or part of an immunoglobulin heavy chain derived from any antibody isotype, e.g. IgG, IgA, IgE and IgM and any of the isotype sub-classes. Fc regions such as Δnab and Δnac as disclosed in WO99/58572 may be employed.

WO 94/25591 discusses the utility of framework regions of immunoglobulins from Camelidae in the provision of single chain binding domains. On other embodiments the antibody or framework regions may be derived from the immunoglobulin of a cartilaginous fish such as a shark (see e.g. J. Immunol. 2008 Jun. 1; 180(11):7461-70)

An antibody molecule in some preferred embodiments of the invention is a monomeric fragment, such as F(ab) or scFv. Such antibody fragments may have the advantage of a relatively short half life.

In addition to antibody sequences, an antibody molecule according to the present invention may comprise other amino acids, e.g. forming a peptide or polypeptide, such as a folded domain, or to impart to the molecule another functional characteristic (e.g. improved half-life) in addition to ability to specifically bind ICAM-3.

In one embodiment, antibody molecules of the invention may be modified with hydrophilic moieties, particularly a polyethylene glycol (PEG) moiety, wherein said hydrophilic moiety is covalently bound to each terminus through an amino acid such as, for example, lysine or any other suitable amino acid or amino acid analogue capable of serving as a linker molecule; and isolating the antibody.

Those skilled in the art are aware of numerous approaches to chemically conjugating molecules to proteins. When the antibody molecule is for pharmaceutical use the conjugate bond is preferably stable in circulation but labile once the conjugate is sequestered intracellularly.

Thus, for example, antibody molecules of the invention may be labelled with a detectable or functional label. Detectable labels include radiolabels such as ¹³¹I or ⁹⁹Tc, which may be attached to antibodies of the invention using conventional chemistry known in the art of antibody imaging. Labels also include enzyme labels such as horseradish peroxidase. Labels further include chemical moieties such as biotin which may be detected via binding to a specific cognate detectable moiety, e.g. labelled avidin. Preferably the labels include fluorescent labels such as FITC.

The present invention further provides an isolated nucleic acid encoding an antibody molecule of the present invention. Nucleic acid includes DNA and RNA. In a preferred aspect, the present invention provides a nucleic acid which codes for a CDR, VH or VL domain of the invention as defined herein, and methods of preparing an antibody molecule, a VH domain and/or a VL domain of the invention, which comprise expressing said nucleic acid under conditions to bring about production of said antibody molecule, VH domain and/or VL domain, and recovering it.

The present invention also provides constructs in the form of plasmids, vectors, transcription or expression cassettes which comprise at least one polynucleotide as above.

The present invention also provides a recombinant host cell which comprises one or more constructs as above. A nucleic acid encoding any CDR, VH or VL domain, or antibody molecule as provided itself forms an aspect of the present invention, as does a method of production of the encoded product, which method comprises expression from nucleic acid which encodes it. Expression may conveniently be achieved by culturing under appropriate conditions recombinant host cells containing the nucleic acid. Following production by expression a VH or VL domain, or antibody molecule may be isolated and/or purified using any suitable technique, then used as appropriate.

Antibody molecules, VH and/or VL domains, and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid according to the present invention may comprise DNA or RNA and may be wholly or partially synthetic.

Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NS0 mouse melanoma cells, YB2/0 rat myeloma cells and many others. A common, preferred bacterial host is E. coli.

The expression of antibodies and antibody fragments in prokaryotic cells such as E. coli is well established in the art. For a review, see for example Plückthun, A. Bio/Technology 9: 545-551 (1991). Expression in eukaryotic cells in culture is also available to those skilled in the art as an option for production of an antibody molecule, see for recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992.

Thus, a further aspect of the present invention provides a host cell containing or transformed with nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

The introduction may be followed by causing or allowing expression from the nucleic acid, e.g. by culturing host cells under conditions for expression of the gene.

In one embodiment, the nucleic acid of the invention is integrated into the genome (e.g. chromosome) of the host cell. Integration may be promoted by inclusion of sequences which promote recombination with the genome, in accordance with standard techniques.

The present invention also provides a method which comprises using a construct as stated above in an expression system in order to express an antibody molecule or polypeptide as above.

Thus, for example, the present invention provides in various aspects:

A nucleic acid comprising a nucleotide sequence encoding a VL region exhibiting an amino acid sequence that is 85%, 86%, 87%, 88%, 89%, 90%, 91%, 92%, 93%, 94%, 95%, 96%, 97%, 98% or 99% identical to the MA4 VL sequence or a functional part thereof comprising at least one, particularly at least two, more particularly at least 3 of the light chain CDRs, but especially all CDRs embedded in their natural framework regions.

A nucleic acid comprising a nucleotide sequence encoding a VH region exhibiting an amino acid sequence that is 85%, 86%, 87%, 88%, 89%, 90%, 91%, 92%, 93%, 94%, 95%, 96%, 97%, 98% or 99% identical to the MA4 VH sequence or a functional part thereof comprising at least one, particularly at least two, more particularly at least 3 of the heavy chain CDRs, but especially all CDRs embedded in their natural framework regions.

### Treatment Methods and Materials

Antibody molecules and other agents according to the invention may be used in a method of treatment or diagnosis of the human or animal body, such as a method of treatment (which may include prophylactic treatment) of a disease or disorder in a human patient which comprises administering to said patient an effective amount of the agent. Conditions treatable in accordance with the present invention include those discussed elsewhere herein.

Further aspects of the invention provide methods of treatment comprising administration of an antibody molecule as provided, pharmaceutical compositions comprising such an antibody molecule, and use of such an antibody molecule in the manufacture of a medicament for administration, for example in a method of making a medicament or pharmaceutical composition comprising formulating the antibody molecule with a pharmaceutically acceptable excipient.

Thus in different aspects of the invention, functional blockade of the chemoattractant activity of ICAM-3 using agents or the invention (selective for ICAM-3) can be used for therapeutic effect.

In accordance with the present invention, compositions provided may be administered to individuals. Administration is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors.

Appropriate doses of antibody are well known in the art; see Ledermann J. A. et al. (1991) Int. J. Cancer 47: 659-664; Bagshawe K. D. et al. (1991) Antibody, Immunoconjugates and Radiopharmaceuticals 4: 915-922. The precise dose will depend upon a number of factors, including whether the antibody is for diagnosis or for treatment, the size and location of the area to be treated, the precise nature of the antibody (e.g. whole antibody, fragment or diabody), and the nature of any detectable label or other molecule attached to the antibody. A typical antibody dose will be in the range 25 mg-5.0 g, and this may be administered as a bolus intravenously. The amount used will depend upon which specific agent is used. More preferably, the daily dose is between 0.5 mg/kg of body weight and 15 mg/kg of body weight, more preferably the antibody is administered to the patient intravenously at a dose of from 1.5 to about 15 mg/kg. An ICAM-3 binding agent that is an antibody can thus be administered to the patient intravenously at a dose of 1.5 mg/kg to about 12 mg/kg, 1.5 mg/kg to about 15 mg/kg, 2.5 mg/kg to about 12 mg/kg, or 2.5 mg/kg to about 12 mg/kg. A ICAM-3 binding agent that is an anti-ICAM-3 antibody fragment or other ICAM-3 binding protein can be administered in a dosage equivalent to a dose of 1.5 mg/kg to about 12 mg/kg, 1.5 mg/kg to about 15 mg/kg, 2.5 mg/kg to about 12 mg/kg, or 2.5 mg/kg to about 12 mg/kg of intact antibody.

Modes of administration include intravenous infusion over several hours, to achieve a similar total cumulative dose. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician.

The agents may also be incorporated within a slow or delayed release device. Such devices may, for example, be inserted on or under the skin and the agent may be released over weeks or even months. Such a device may be particularly useful for chronically ill patients. The devices may be particularly advantageous when an agent is used which would normally require frequent administration (e.g. at least daily ingestion of a tablet or daily injection)

More preferably the mode of administration employs pre-coating of, or otherwise incorporation into, indwelling devices, for which the optimal amount of antibody will be determined by means of appropriate experiments. Such embodiments are preferred where a high localised concentration is required.

Agents such as antibody molecules of the present invention will usually be administered in the form of a pharmaceutical composition, which may comprise at least one component in addition to the antibody molecule.

Thus pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A composition may be administered alone or in combination with other treatments, either simultaneously or sequentially dependent upon the condition to be treated. Other treatments may include the administration of suitable doses of pain relief drugs such as non-steroidal anti-inflammatory drugs (e.g. aspirin, ibuprofen or ketoprofen) or opiates such as morphine, or anti-emetics.

### Diseases Susceptible to Treatment by the Present Invention

### Diseases Characterised by Inflammation and Granulomas:

Arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, including chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), and IBD with co-segregate of pyoderma gangrenosum, erythema nodosum, primary sclerosing cholangitis, and/or episcleritis, respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's) Arteritis), Medium Vessel vasculitis (including Kawasaki's Disease and Polyarteritis Nodosa), CNS vasculitis, and ANCA-associated vasculitis, such as Churg-Strauss vasculitis or syndrome (CSS)), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic thrombocytopenic purpura (TTP), thrombocytopenia (as developed by myocardial infarction patients, for example), including autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barré Syndrome, Berger's Disease (IgA nephropathy), primary biliary cirrhosis, celiac sprue (gluten enteropathy), refractory sprue with co-segregate dermatitis herpetiformis, cryoglobulinemia, amylotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

### Cancers:

Fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, rectal cancer, colorectal cancer, kidney cancer, pancreatic cancer, bone cancer, breast cancer, ovarian cancer, prostate cancer, penile carcinoma, esophogeal cancer, gastric cancer, gastrointestinal cancer, stomach cancer, peritoneal cancer, hepatic carcinoma, hepatocellular cancer, liver cancer, oral cancer, nasal cancer, throat cancer, squamous cell carcinoma (e.g., epithelial), basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, endometrial or uterinecarcinoma, vulval cancer, testicular cancer, bladder carcinoma, lung cancer, including small cell lung carcinoma, non-small cell lung cancer, adenocarcinoma, of the lung and squamous carcinoma of the lung, epithelial carcinoma, glioma, glioblastoma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma, salivary gland carcinoma, thyroid cancer, head cancer, neck cancer, anal cancer, Blood-borne cancers, including, but not limited to: acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, myelodysplastic syndromes “MDS”, Acute and chronic leukemias: lymphoblastic, myelogenous, lymphocytic, myelocytic leukemias, Lymphomas: Hodgkin's disease, non-Hodgkin's Lymphoma, Multiple myeloma, Waldenström's macroglobulinemia, Heavy chain disease, Polycythemia vera,

### TERMINOLOGY

### Antibody Molecule

The terms “Antibody molecule” as used herein is understood to refer to molecules or active fragments of molecules that bind to known antigens, particularly to refer to immunoglobulin molecules and to immunologically active portions of immunoglobulin molecules, i.e. molecules that contain a binding site that immunospecifically binds an antigen. An immunoglobulin according to the invention can be of any type (IgG, IgM, IgD, IgE, IgA and IgY) or class (IgG1, IgG2, IgG3, IgG4, IgA1 and IgA2) or subclasses of immunoglobulin molecule.

Antibodies molecules may be natural or partly or wholly synthetically produced.

Antibodies that are intended to be within the scope of the present invention include monoclonal, polyclonal, chimeric, single chain, bispecific or bi-effective, simianized, human and humanized antibodies as well as active fragments thereof. Examples of active fragments of molecules that bind to known antigens include (which comprise an antigen binding domain) include Fab, F(ab′)₂, scFv, Fv, and the products of an Fab immunoglobulin expression library, and epitope-binding fragments of any of the antibodies and fragments, plus also dAb, Fd; diabodies and so on.

Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al, Proc. Natl. Acad. Sci. USA 90, 6444-6448, 1993). Fv, scFv or diabody molecules may be stabilised by the incorporation of disulphide bridges linking the VH and VL domains (Y. Reiter et al, Nature Biotech, 14, 1239-1245, 1996). Minibodies comprising a scFv joined to a CH3 domain may also be made (S. Hu et al, Cancer Res., 56, 3055-3061, 1996).

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against ICAM-3, then a library can be made where the other arm is varied and an antibody of appropriate specificity selected. Bispecific whole antibodies may be made by knobs-into-holes engineering (J. B. B. Ridgeway et al, Protein Eng., 9, 616-621, 1996).

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

### Antigen Binding Domain

Where used herein this describes the part of an antibody molecule which comprises the area which specifically binds to and is complementary to part or all of an antigen. Where an antigen is large, an antibody may only bind to a particular part of the antigen, which part is termed an epitope.

“Specific binding” in this context will be understood to relate to binding arising from a specific interaction between the conformation of an antigen binding domain and its binding partner, as opposed to non-specific binding arising only from van der Waals forces or other non-specific protein:protein interactions.

### CDR

The term “CDR” refers to the hypervariable region of an antibody. The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions are based on sequence variability and are the most commonly used (Kabat et al., Sequences of Proteins of Immunological Interest, 5th Ed. Public Health Service, National Institutes of Health, Bethesda, Md. (1991)).

The structure for carrying a CDR of the invention will generally be of an antibody heavy or light chain sequence or substantial portion thereof in which the CDR is located at a location corresponding to the CDR of naturally occurring VH and VL antibody variable domains encoded by rearranged immunoglobulin genes.

Variable domains employed in the invention may be obtained from any germ-line or rearranged human variable domain, or may be a synthetic variable domain based on consensus sequences of known human variable domains. A CDR sequence of the invention (e.g. CDR3) may be introduced into a repertoire of variable domains lacking a CDR (e.g. CDR3), using recombinant DNA technology.

A further alternative is to generate novel VH or VL regions carrying a CDR-derived sequences of the invention using random mutagenesis of one or more selected VH and/or VL genes to generate mutations within the entire variable domain. Such a technique is described by Gram at al (1992, Proc. Natl. Acad. Sci., USA, 89:3576-3580), who used error-prone PCR.

### Humanized Antibody

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl. Acad Sci USA, 86:10029-10032 (1989), Hodgson et al., Bio/Technology, 9:421 (1991)).

A “humanized antibody” may also be obtained by a novel genetic engineering approach that enables production of affinity-matured humanlike polyclonal antibodies in large animals such as, for example, rabbits (see, e.g. U.S. Pat. No. 7,129,084).

### Monoclonal Antibody

The term “monoclonal antibody” is also well recognized in the art and refers to an antibody that is mass produced in the laboratory from a single clone and that recognizes only one antigen. Monoclonal antibodies are typically made by fusing a normally short-lived, antibody-producing B cell to a fast-growing cell, such as a cancer cell (sometimes referred to as an “immortal” cell). The resulting hybrid cell, or hybridoma, multiplies rapidly, creating a clone that produces large quantities of the antibody. For the purpose of the present invention, “monoclonal antibody” is also to be understood to comprise antibodies that are produced by a mother clone which has not yet reached full monoclonality.

### Functionally Equivalent Antibody

“Functionally equivalent antibody” is understood within the scope of the present invention to refer to an antibody which substantially shares at least one major functional property with MA4 for example functional properties herein described including, but not limited to: binding specificity to ICAM-3.

### Immunogen and Antigen

An “immunogen” is defined as any substance that can induce an adaptive immune response whereas an “antigen” is any substance that can be recognised (in terms of an immune response) by the cells of the adaptive immune system.

### Comprise

This is generally used in the sense of “include”, that is to say permitting the presence of one or more features or components.

### Isolated

This refers to the state in which antibody molecules of the invention, or nucleic acid encoding such antibody molecules, will generally be in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Antibody molecules may be glycosylated, either naturally or by systems of heterologous eukaryotic cells (e.g. CHO or NS0 (ECACC 85110503) cells, or they may be (for example if produced by expression in a prokaryotic cell) unglycosylated.

### Variant Sequences

By “substantially as set out” it is meant that the relevant CDR or VH or VL domain of the invention will be either identical or highly similar to the specified regions of which the sequence is set out herein. By “highly similar” it is contemplated that from 1 to 5, preferably from 1 to 4 such as 1 to 3 or 1 or 2, or 3 or 4, amino acid substitutions may be made in the CDR and/or VH or VL domain.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for example 95% identity with a reference sequence of the present invention, the parameters are preferably adjusted so that the percentage of identity is calculated over the entire length of the reference sequence and homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

As used herein a “conservative change” refers to alterations that are substantially conformationally or antigenically neutral, producing minimal changes in the tertiary structure of the mutant polypeptides, or producing minimal changes in the antigenic determinants of the mutant polypeptides, respectively, as compared to the native protein. When referring to the antibodies and antibody fragments of the invention, a conservative change means an amino acid substitution that does not render the antibody incapable of binding to the subject epitope. One of ordinary skill in the art will be able to predict which amino acid substitutions can be made while maintaining a high probability of being conformationally and antigenically neutral. Such guidance is provided, for example in Berzofsky, (1985) Science 229:932-940 and Bowie et al. (1990) Science 247:1306-1310. Factors to be considered that affect the probability of maintaining conformational and antigenic neutrality include, but are not limited to: (a) substitution of hydrophobic amino acids is less likely to affect antigenicity because hydrophobic residues are more likely to be located in a protein's interior; (b) substitution of physiochemically similar, amino acids is less likely to affect conformation because the substituted amino acid structurally mimics the native amino acid; and (c) alteration of evolutionarily conserved sequences is likely to adversely affect conformation as such conservation suggests that the amino acid sequences may have functional importance. One of ordinary skill in the art will be able to assess alterations in protein conformation using well-known assays, such as, but not limited to microcomplement fixation methods (see, e.g. Wasserman et al. (1961) J. Immunol. 87:290-295; Levine et al. (1967) Meth. Enzymol. 11:928-936) and through binding studies using conformation-dependent monoclonal antibodies (see, e.g. Lewis et al. (1983) Biochem. 22:948-954).

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1

Identification & Characterisation of ICAM-3 Reactive mabs
A. Supernatants from hybridomas MA4, GD12 and BH10 were tested for reactivity against ICAM-3. Domain 1-2 ICAM-3-Fc was immobilised to an ELISA plate using anti-human Fc antiserum. Reactivity of indicated mAbs was detected using anti-mouse-HRP. Data are presented as mean±SD of triplicate wells.
B. Western blot lysates of HEK cells transiently expressing ICAM-3 and their mock-transfected counterparts probed with mAbs MA4, GD12 and BH10.
C. Flow cytometric immunofluorescence histograms of ICAM-3-transfected HEK cells (open plots) or their mock-transfected counterparts (shaded plots) stained with MA4 and GD12.

FIG. 2

Inhibition of apoptotic cell interaction with THP-1 phagocytes by anti-ICAM-3 mAb MA4. UV-induced apoptotic BL cells were co-cultured with THP-1 phagocytes in the presence of the indicated mAbs. (A) the proportion of phagocytes interacting with apoptotic cells was scored using light microscopy. Data (mean±SD) from at least three separate experiments each carried out in quadruplicate are shown (data is normalised to the mAb-free co-culture (Alone); (B) Microscopic appearance of co-cultures. Scale bar=50 μm. *P<0.05. Mann-Whitney test.

FIG. 4

Apoptotic cell ICAM-3 mediates tethering to macrophages at different temperatures. The results with wild-type are shown on the left hand side of each of the pair.

FIG. 5

Apoptotic ICAM-3 functions independently of CD14. The order of the bars under each set of conditions is 0 mAb, +MA4, +61 D3.

FIG. 6

Loss of ICAM-3 expression occurs early in apoptosis.

FIG. 7

Microparticles containing ICAM-3 promote chemoattraction.

FIG. 8

ICAM-3 reactive mAb inhibition of chemoattraction.

FIG. 9

Sequence alignment of ICAM-1, ICAM-2, and ICAM-3. Sequence alignment of the two N-terminal domains of ICAM-1, ICAM-2, and ICAM-3. The predicted b strands of the Ig domains are underlined and labelled according to the method of Casanovas et al. (25), as defined for ICAM-2. Asterisks denote those residues in ICAM-3 that were targeted for site-directed mutagenesis. Potential N-linked glycosylation sites are numbered (−1—etc.) above the alignment (from J. Immunol. 1998; 161; 1363-1370; Elaine D. Bell, Andrew P. May and David L. Simmons Domain).

FIG. 10

Microparticles from apoptotic cells mediate macrophage chemoattraction in an ICAM-3 dependent manner: Microparticles were prepared from cultures of apoptotic Mutu BL cells (WT or ICAM-3-deficient) at 16 h post-UV. Supernatants were centrifuged to remove cell bodies (7 min; 350×g) and used neat. (a) Chemotaxis assay: the bottom chamber contained microparticles whilst the top chamber contained dihydroxyvitamin D3 stimulated THP-1 cells. Migration was allowed to proceed for 4 hours prior to staining of migrated cells and quantitation by light microscopy. For each experimental treatment 4 replicate wells were used and within each 5 high power fields (hpf) of view counted to quantify the number of migrated cells. Data shown are mean±SE of independent experiments (n=7)*P<0.05 ANOVA with Tukey's post-hoc test. (b) Chemokinesis control where the chemotactic gradient was disrupted via inclusion of microparticles in both upper and lower chambers. Data shown are mean±SE of independent experiments (n=4)*P<0.05 ANOVA with Tukey's post-hoc test.

### EXAMPLES

### General Methods and Materials

### Cell Isolation, Cell Lines and Culture

Anti-ICAM-3 expressing hybridoma cells were produced by fusing spleen cells from Balb/c mice immunised with ICAM-3-Fc ((17)) with Ns0 cells. Briefly, spleen cells were harvested following mechanical spleen disruption and mixed with Ns0 myeloma cells (5:1 ratio). Cells were pelleted using centrifugation (5 min, 200 g), supernatant removed and cells gently resuspended in 50% v/v PEG 1500 (Roche) at 37° C. Following 2 min incubation at 37° C., 13 ml RPMI was added drop-wise with swirling prior to a further 15 min incubation. Cells were then placed into medium (Iscove's modified Dulbecco's Medium+20% FCS; all PAA, UK) before cloning by limiting dilution in the presence of HAT (Sigma) as a selective agent.

All work was undertaken in accordance with regulatory guidance. Hybridomas were cloned using limiting dilution in RPMI 1640 medium containing 2 mM L-glutamine supplemented with 10% Foetal calf serum (PAA, Yeovil, UK) and 100 IU ml⁻¹ penicillin and 100 μg ml⁻¹ streptomycin). ICAM-3 reactivity was assessed using ELISA of culture supernatants against immobilised ICAM-3-Fc and CD14-Fc.

Mutu I Burkitt lymphoma (BL) cells (24), Jurkat (human T) (25) and THP-1 (human myelomonocytic (26)) were cultured continuously in RPMI 1640 medium containing 2 mM L-glutamine supplemented with 10% Foetal calf serum (PAA, Yeovil, UK) and 100 IU ml⁻¹ penicillin and 100 μg ml⁻¹ streptomycin). RAW 264.7 cells (murine macrophage (27)), J774 cells (murine macrophage (28)), HeLa 229 (human epithelial) and HEK-293 cells (human epithelial) cells were cultured similarly but in DMEM (PAA, Yeovil, UK).

ICAM-3-deficient Mutu I cells were generated by sequential fluorescence-activated cell sorting of a wild-type Mutu I population. Briefly, cells were stained aseptically with excess anti-ICAM-3 mAb (CAL3.10; R&D Systems) followed by goat anti-mouse-FITC (Sigma). At least 10,000 viable cells from within the lowest 5% fluorescence region were sorted using a Beckman-Coulter cell sorter. Once cultured for 1-2 weeks this process was repeated twice to produce stable, non-ICAM-3 expressing cell populations.

THP-1 cells were differentiated to macrophages by stimulation with dihydroxyvitamin D3 (100 nM) for 48-72 hours.

### Monoclonal Antibody-Based Assays

mAbs were produced as outlined above and clones of interest were further cultured and tissue culture supernatant harvested from static cultures when cells were 70+% apoptotic. Such tissue culture supernatants were routinely for subsequent analyses.

For ELISA, Nuclon Maxisorop plates (Nunc) were coated overnight with sheep-anti-human Fc (The Binding Site) at 5 μg/ml in carbonate buffer (pH 9.6; Sigma). Following washing in PBS(T), Fc-tagged proteins were added a concentration of 1 μg/ml for 1 hour at 37° C. The resultant, oriented Fc-fusions were probed with mAbs and bound mAb detected using bound mAb was detected using anti-mouse-HRP (GE Healthcare) and colorimetric SigmaFAST OPD assay (Sigma).

For Western blot analysis, cells were solubilised under reducing and denaturing conditions, separated using standard polyacrylamide gel electrophoresis and electroblotted to nitrocellulose (0.45 μm Schleicher & Schuell). Membranes were blocked in 5% non-fat milk powder in TBS-tween (0.05% v/v; pH7.2), probed with mAbs and detected using anti-mouse HRP and Amersham ECL detection kits.

For indirect immuno-fluorescence of cells, cells were incubated with excess mAb on ice for 15-30 minutes, washed in 0.5% (w/v) in BSA in PBS(A) and incubated with goat-anti mouse-FITC (1 μl per 200,000 cells in 100 μl volume; Sigma). Stained cells were analysed either directly or following fixation in 1% w/v formaldehyde in PBS using a Beckman-Coulter Quanta SC or Beckman-Coulter EPICS-XL flow cytometer (Beckman Coulter, High Wycombe, UK). Downstream flow cytometric analyses and presentations were undertaken using VenturiOne software from Applied Cytometry Systems (Sheffield, UK) or FlowJo (Treestar Inc, USA).

### Production and Binding of Soluble Recombinant Human CD14 and ICAM-3

Recombinant human CD14 or human ICAM-3 were produced as Fc fusion proteins (fused to CH2/CH3 domains of human IgG1). DNA for the recombinant proteins was transfected to 293 cells using standard calcium phosphate mediated transfections. Fc-fusion proteins were purified from culture supernatants using HiTrap protein G columns (GE Healthcare). For staining 200,000 cells were incubated with 1 μg of CD14-Fc; ICAM-3-Fc or human IgG (The Binding Site) for 30 min at 4° C. Cell-associated Fc-containing proteins were detected by staining phycoerythrin-conjugated goat-anti-human IgG (Sigma).

### Apoptosis Induction and Quantification

BL or Jurkat cells were subjected to UV-B irradiation (UVP-Chromatovue C71 light box fitted with 315 nm 15 W tubes) to induce apoptosis. Dose was assessed using a digital radiometer (UVP radiometer with mid range sensor calibrated at 310 nm). BL cells received 100 mJ cm⁻² whilst Jurkats received 200 mJ cm⁻². For analysis of apoptotic nuclear morphology, cells were fixed in 1% formaldehyde, stained with 4,6-diamidino-2-phenylindole (DAPI, Sigma, 250 ng ml⁻¹) and observed using inverted epifluorescence microscopy. For quantitative analyses, percentages of apoptotic cells per ≧200 counted per sample were enumerated. Photomicrography was undertaken using a fully motorised Zeiss Axiovert fluorescence microscope (Carl Zeiss Ltd., Welwyn Garden City, UK) and Hamamatsu Orca camera driven by Volocity (Improvision, Coventry, UK).

Apoptotic cells for use in Example 2 onwards were generated as above except: Mutu cells±ICAM-3 were suspended at 2 million per ml in RPMI with 0.1% w/v Bovine serum albumin (BSA). The cells were then irradiated (100 mJ/cm2) and left to die for 16-18 hours.

### Annexin V Labelling and Flow Cytometry

Cells were stained with annexin V-FITC (Lonza Biologics, Slough, UK). Briefly, cells were washed and resuspended in binding buffer (10 mM HEPES, 150 mM NaCl, 2.5 mM CaCl₂) containing annexin V-FITC (1 μl per 200,000 cells) for 2 min on ice. Cells were diluted to 1 ml with binding buffer and PI was added to a final concentration of 20 μg/ml. Samples were analysed immediately on a Quanta SC flow cytometer (Beckman Coulter, High Wycombe, UK).

Assays of Phagocyte Interaction with Apoptotic Cells

Assay of interaction (binding and phagocytosis) of phagocytes with apoptotic cells was carried out either on multi-well glass slides (29) or in 24-well plates as described (30) (31). Briefly, for the slide-based assay, macrophages and apoptotic cells (10⁶ per well) were co-cultured for 1 hr at 37° C. in RPMI containing 0.2% (w/v) bovine serum albumin (Sigma). Unbound cells were removed by extensive washing and slides fixed in methanol, stained with Jenner/Giemsa (Raymond Lamb) and mounted in DePeX (BDH) prior to examination by light microscopy. Assays were performed similarly in 24-well plates but at the end of the procedure, cells were fixed in 1% formaldehyde and stained with DiffQuick II (Dade Diagnostika Gmbtt, D-80807 Munich). In all cases at least 200 macrophages were assessed in each of duplicate wells. As appropriate, antibodies were included at a 1:10 dilution of tissue culture supernatant. For assay of binding co-cultures were carried out at room temperature (20° C.). Data are presented as percent macrophages binding or interacting with apoptotic cells

Where THP-1 cells were used as the phagocyte source cells were cultured for 72 h in the presence of dihydroxyvitamin D3 (Calbiochem, 100 nM) and/or PMA (Sigma, 250 nM) in 24-well plates.

### Chemoattraction Assays

Large cell debris was removed by centrifugation (5 min 200 g) and the remaining supernatant constitutes “neat” i.e. undiluted microparticles. This was used as a putative chemoattractant either neat or diluted (in RPMI/BSA).

Control chemoattractant was RPMI/0.1% w/v BSA alone. On the day of the assay the THP-1 cells were resuspended at 2 million per ml in RPMI/BSA. The assay was set up in a chemotaxis chamber (http://www.neuroprobe.com/products/ap48.html) using 5 μm filters. THP cells in top chamber. Putative chemoattractants in the bottom. Incubation at 37° C. for 4 hours. Cells bound to underside of the membrane (i.e. Those that had crawled through to the bottom side of the chamber) were scored. The nature of the membranes used (PCTE—Neuroprobe Inc) means that the THP cells remained stuck to the membrane and could then be scored using light microscopy following staining of the membrane with DiffQuik II).

### Example 1

### Characterisation of Anti-ICAM-3 mAbs

Moffatt et al. ((17)) characterised anti-ICAM-3 mAbs capable of blocking the interaction of apoptotic cells with phagocytes (BU68 and 3A9) showing that they were reactive to the most membrane-distal domains of ICAM-3 (domains 1-2).

The sequence of these domains are shown in FIG. 9.

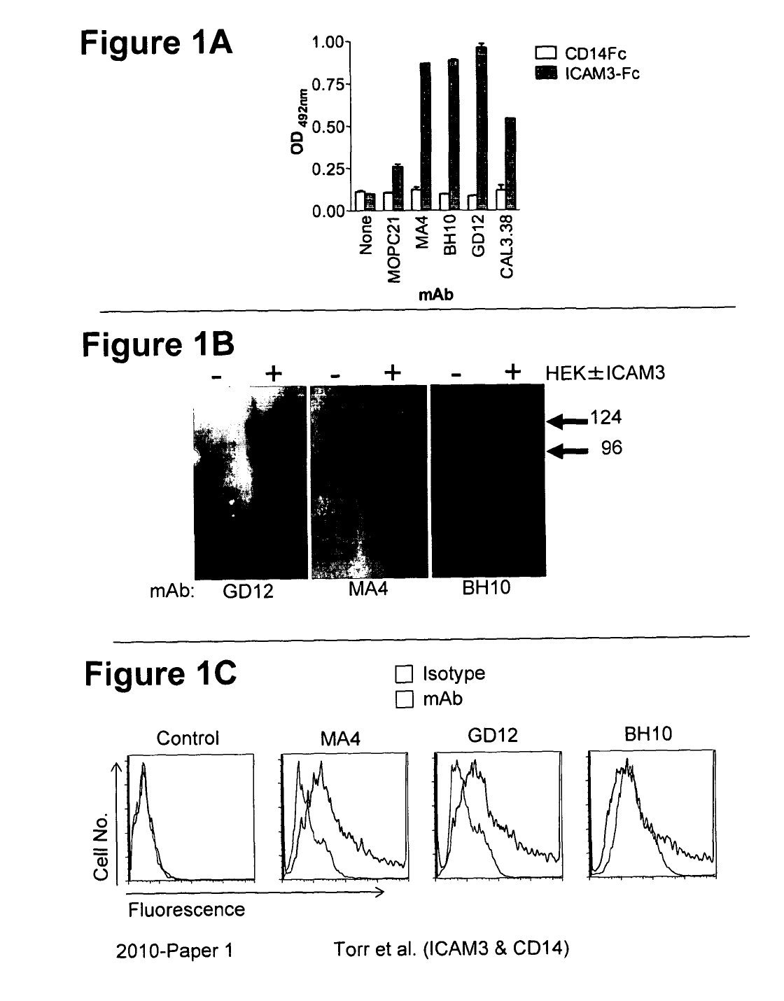

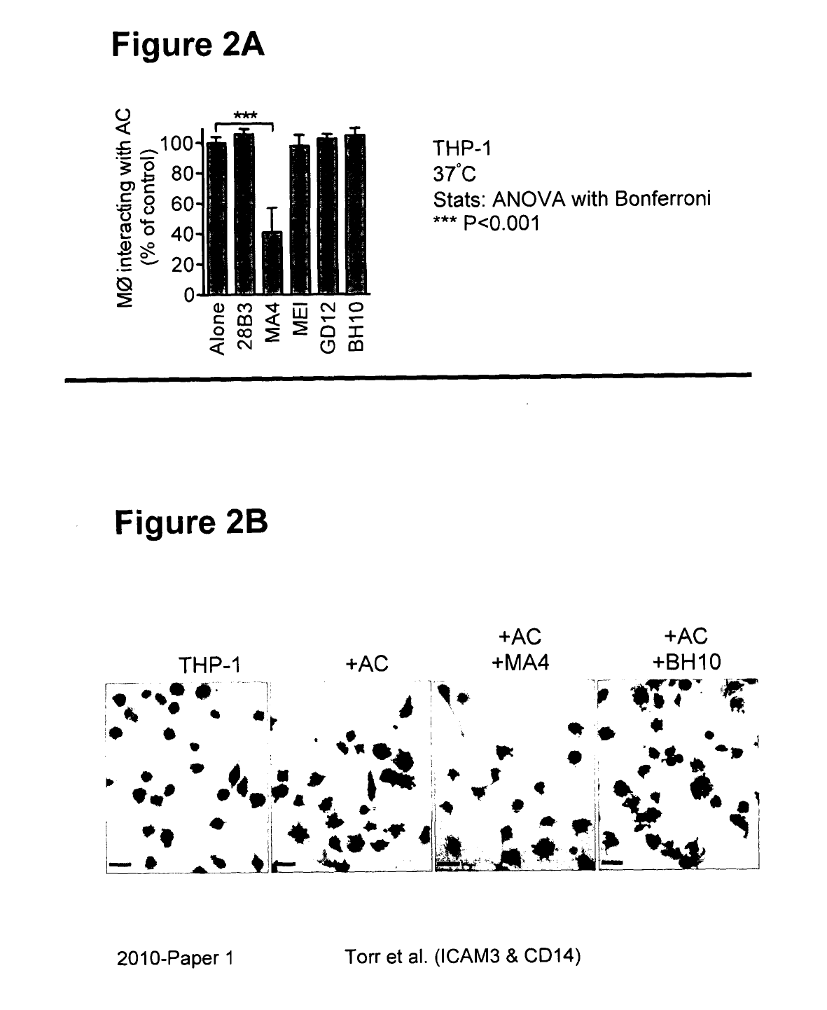

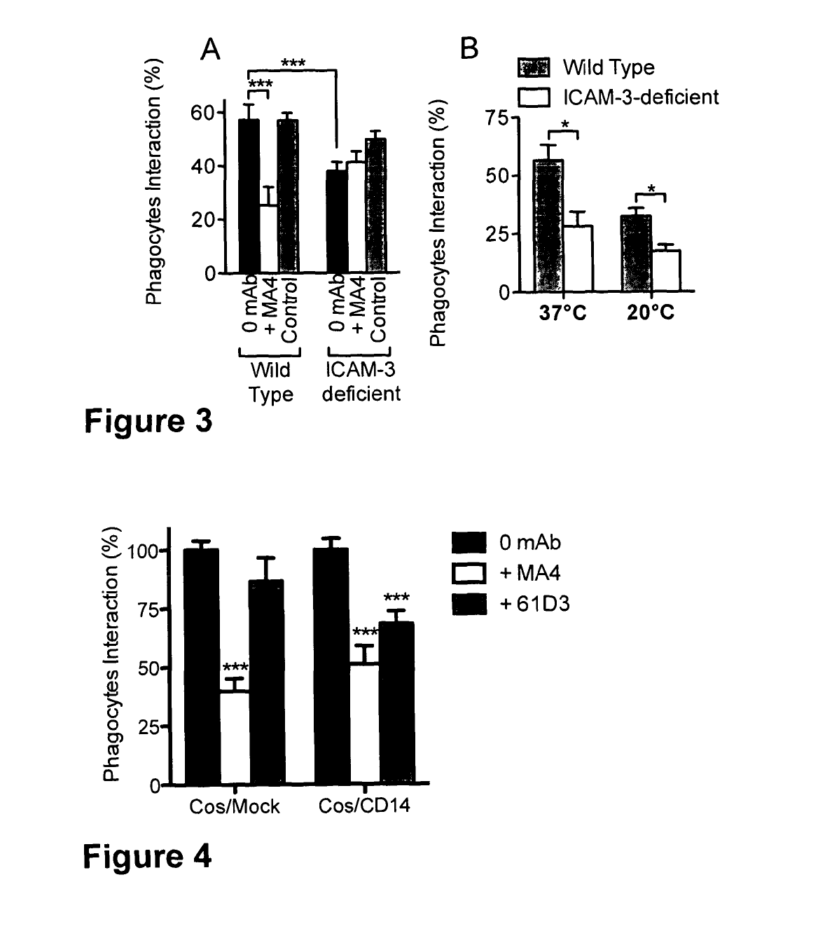

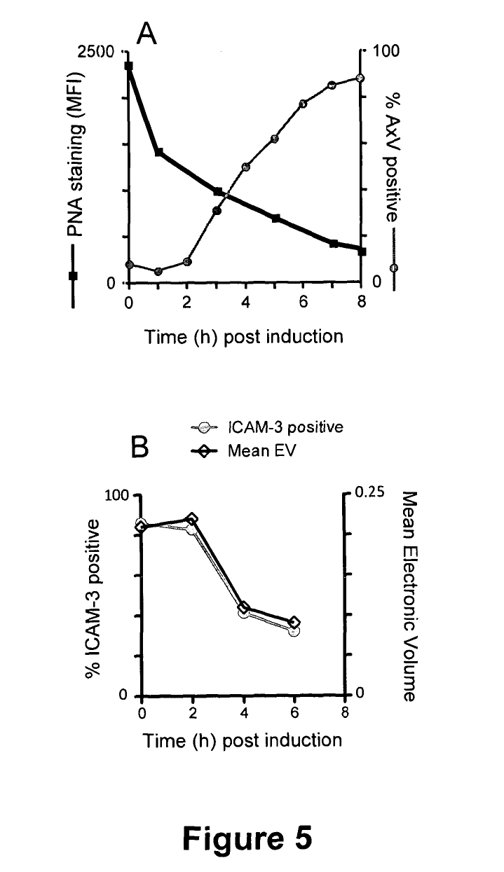

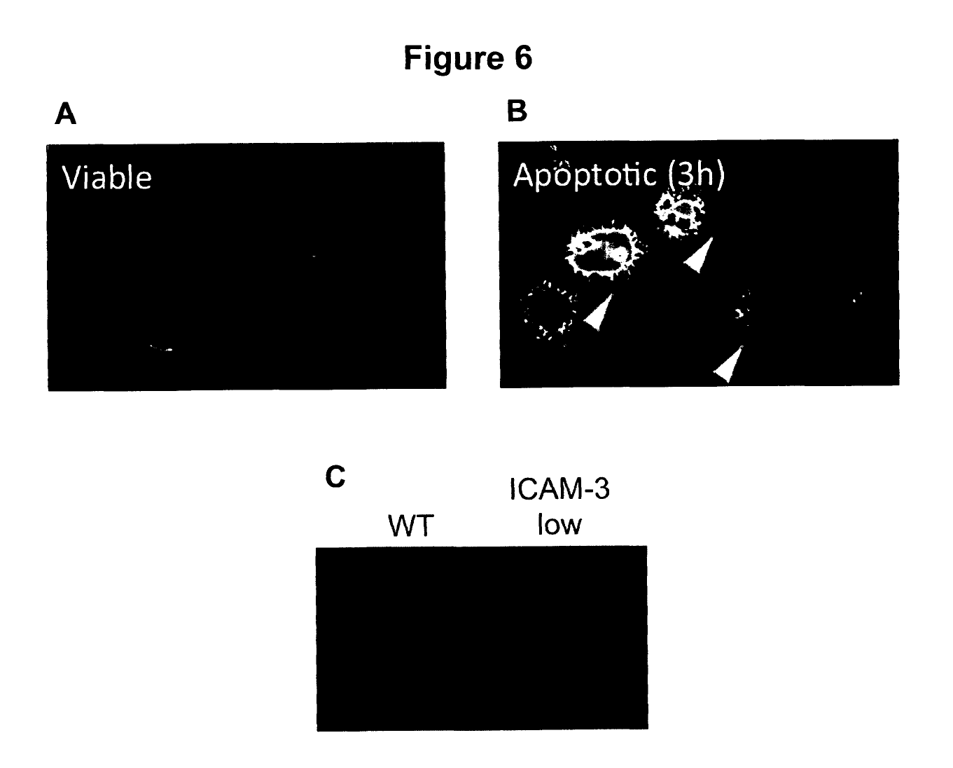

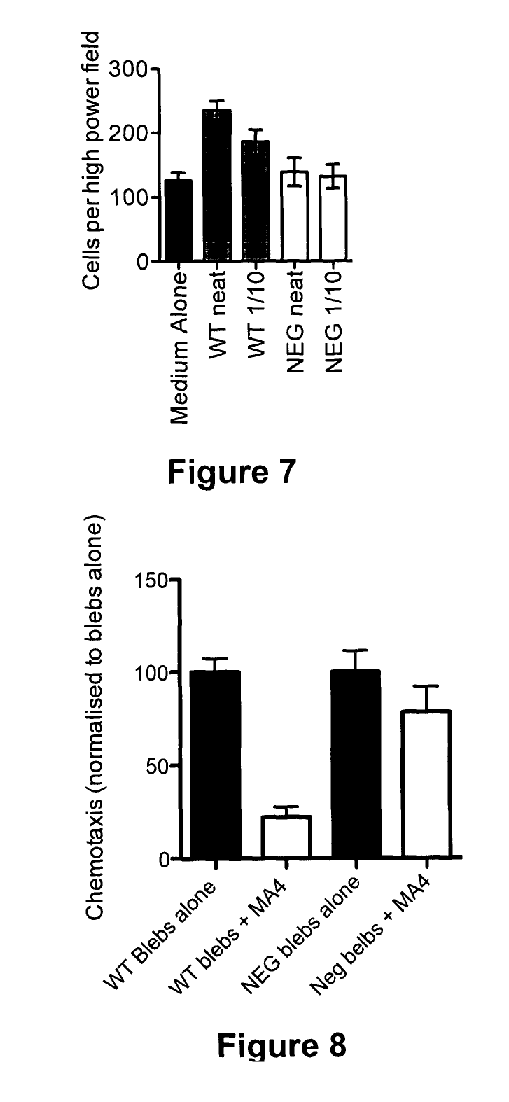

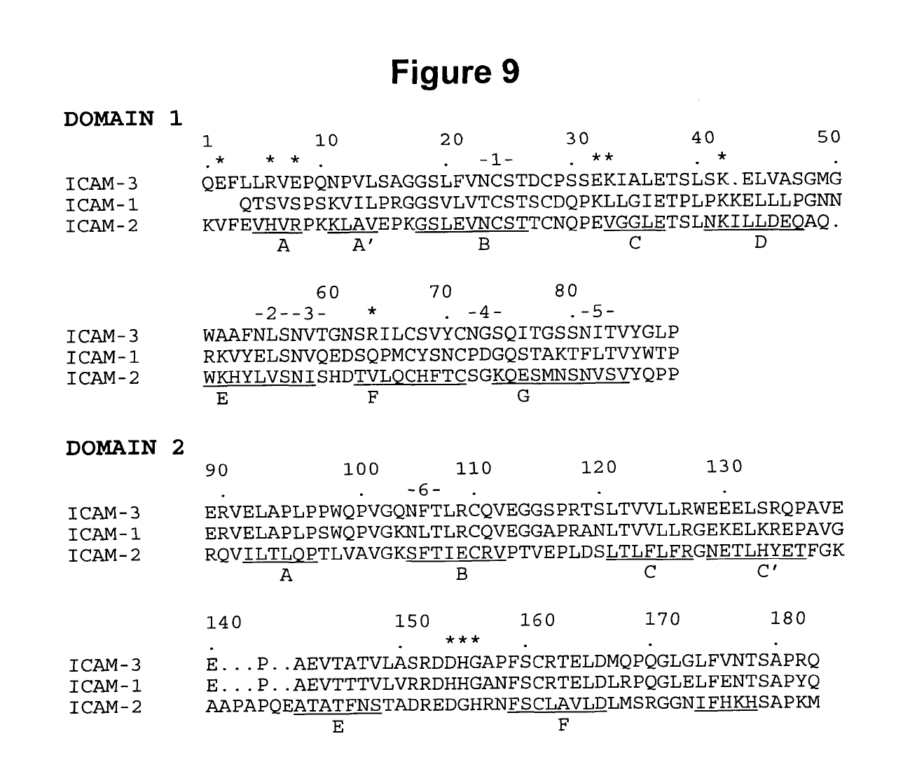

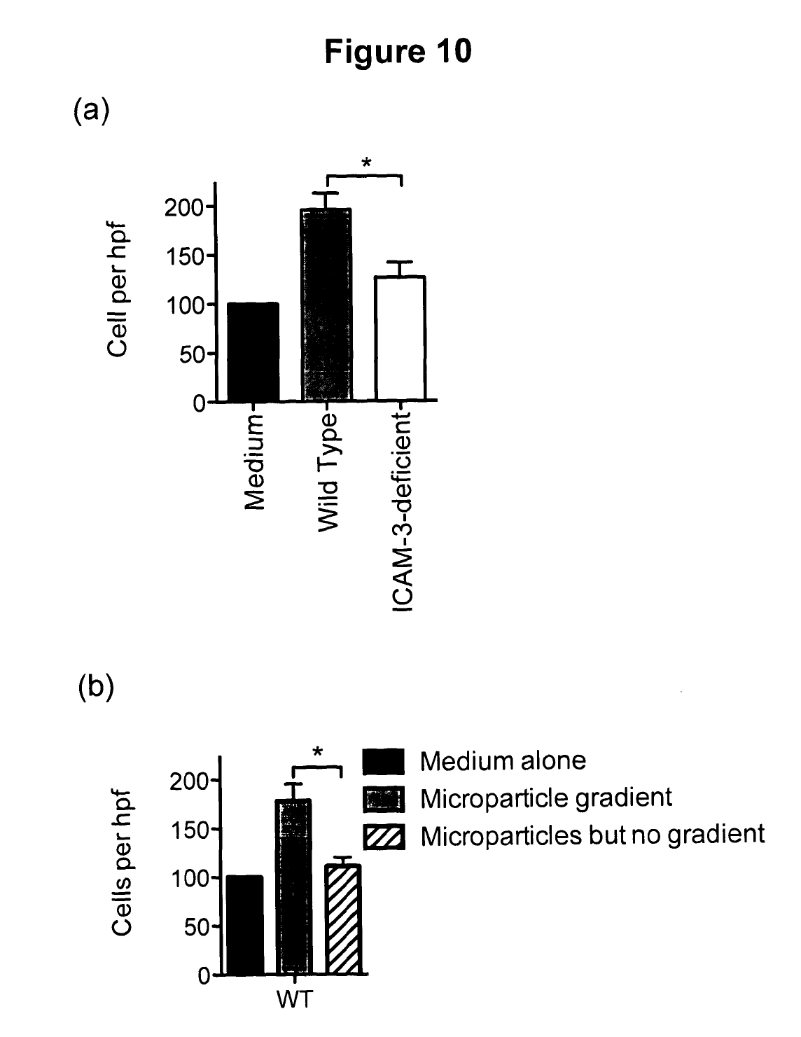

In the present work screens of a panel of novel mAbs raised from mice immunised with ICAM-3-transfected HEK cells were screened by ELISA against immobilised recombinant ICAM-3 (D1-2)-Fc fusion protein and against CD14-Fc as a control. We identified 17 anti-ICAM-3 mAbs and the reactivity of three (MA4, BH10 and GD12) specific for domains 1-2 of ICAM-3 are shown (FIG. 1A). The ability of these mAbs to bind full length, cell-associated ICAM-3 was further tested. Western blot analysis of HEK cells, which naturally lack ICAM-3, and their ICAM-3-transfected counterparts, was undertaken following polyacrylamide gel electrophoresis. These analyses indicated that MA4, BH10 and GD12 were all capable of reacting with full-length (domains 1-5) ICAM-3 that was both denatured and reduced ICAM-3 (FIG. 1B) though a number of other mAbs failed to react suggesting they reacted to a conformational, non-linear epitope. The double band pattern of reactivity seen in these Western blots was characteristic and consistent with our previous analyses ((17)). Additionally flow cytometric analysis of transfected HEK cells was undertaken following indirect immunofluorescence staining of unfixed cells to assess the capability of mAbs to bind to cell surface-associated, full length ICAM-3. All three mAbs tested showed strong ICAM-3-specific staining of ICAM-3 transfectants (FIG. 1C).

Using a model system of specific apoptotic cell clearance ((30)) we screened available anti-ICAM-3 mAbs and identified one, MA4, that robustly inhibited clearance (FIG. 2). The observed degree of inhibition with MA4 was in line with that seen previously with ICAM-3 mAbs BU68 and 3A9 ((17, 32)). Other mAbs that were also screened (e.g. BH10 and GD12) failed to inhibit clearance though they were all of the same isotype (murine IgG1 mAbs bearing a kappa light chain—data not shown).

These results confirm the identification of a novel anti-ICAM-3 mAb (MA4) that reacts with a linear epitope in domain 1-2 of ICAM-3, which is capable of inhibiting apoptotic cell interaction with phagocytes.

### Example 2

### Apoptotic Cell ICAM-3 Mediates Tethering to Macrophages

Human B lymphocyte cell lines (ICAM-3-deficient or Wild Type ICAM-3-replete counterparts) were induced to apoptosis and co-cultured with the phagocytic human MØ-like cell line THP-1.

The results are shown in FIG. 3.

(A) MA4, a novel anti-ICAM-3 mAb, reduces the ability of MO to interact with apoptotic Wild Type but not ICAM-3-deficient cells.

(B) The ability of phagocytes to recognise apoptotic cells at 37° C. (permissive for binding and phagocytosis) and 20° C. (permissive only for binding—see (3)) was assessed using microscopy. At both temperatures, ICAM-3-deficient cells interact with phagocytes less well than their wild-type ICAM-3-replete counterparts.

### Example 3

### Apoptotic ICAM-3 Functions Independently of CD14

Apoptotic human B cells were co-cultured with Cos cells (mock or CD14-transfected) in the presence of mAbs, MA4 or 61D3 (anti-CD14).

As shown in FIG. 4, MA4 inhibits clearance of apoptotic cells irrespective of CD14 expression. Whilst previous work has suggested apoptotic ICAM-3 binds to the MØ receptor CD14⁵ these data indicate that other receptors are likely to be involved. This is supported by our observations that soluble CD14 binds equally to apoptotic cells irrespective of their ICAM-3 expression (data not shown).

### Example 4

### Loss of ICAM-3 Expression Occurs Early in Apoptosis

FIG. 5 shows that changes in human B cell surface glycosylation occur early in apoptosis and precede changes in annexin V staining (A×V). Alterations in ICAM-3 levels (detectable with mAb staining) are closely associated with changes in cell size (as assessed by flow cytometry—electronic volume) and occur very early in apoptosis. (A) PNA (an ICAM-3-reactive lectin) and in V staining; (B) Flow cytometric data of ICAM-3 versus electronic volume.

### Example 5

### ICAM-3 is Released from Apoptotic Cells

FIG. 6 shows HeLa cells transfected with ICAM-3-GFP (A) were induced to apoptosis (B). ICAM-3 (green) along with DNA (blue) distributes to apoptotic bodies (arrows). Western blot analysis (C) of apoptotic cell supernatants of human B cell lines (ICAM-3-deficient or Wild Type) demonstrates ICAM-3 release in microparticles accounting for reduced ICAM-3 expression during apoptosis.

### Example 6

### Microparticles Containing ICAM-3 Promote True Chemoattraction

Microparticles were purified from supernatants of apoptotic human B cells (ICAM-3-deficient or Wild Type) and their chemoattractive capacity (neat and 1/10 dilution) assessed using chemotaxis chambers (5 μm). As shown in FIG. 7, microparticles containing ICAM-3 were potent chemo-attractants for MO. This finding was confirmed in further experiments, with the results being shown in FIG. 10(a).

FIG. 10(b) shows that when MØ are exposed to the ICAM-3 microparticles in the absence of a gradient, they did not move. This confirms the findings herein that the ICAM-3 on the microparticles is causing true directional chemotaxis i.e. movement up a gradient,

### Example 7

### mAb Inhibition of Chemoattraction

THP-1 cells were stimulated to differentiate with dihydroxyvitamin D3 for 48 hours. Microparticles from B cells with or without ICAM-3 were prepared. mAb (anti-ICAM-3) was added as a 1/10 dilution of tissue culture supernatant of the mAb producing hybridoma.

ICAM-3 replete blebs are inhibited in the chemoattractive property by the ICAM-3 mAB whilst the ICAM-3-negative blebs are not.

Data is presented in FIG. 8 as percentage of “microparticles alone” to show the extent of inhibition. mean±SEM from a single experiment is shown. This is representative of 2 similar experiments.

### REFERENCES

1. Gregory, C. D. & Devitt, A. (2004) Immunology, 113, 1-14.
2. Devitt, A., et al. (1998): Nature, 392, 505-9
3. Devitt, A., et al (2004). JCB 167, 1161-70.
4, Devitt, A. et al. (2003). Cell Death & Diff 10, 371-382
5\17. Moffatt O D, Devitt A, Bell E D, Simmons D L, Gregory C D. Macrophage recognition of ICAM-3 on apoptotic leukocytes. J Immunol 1999 Jun. 1; 162 (11): 6800-6810.
25. Schneider U, Schwenk H U, Bornkamm G. Characterization of EBV-genome negative “null” and “T” cell lines derived from children with acute lymphoblastic leukemia and leukemic transformed non-Hodgkin lymphoma. Int J Cancer 1977 May 15; 19 (5): 621-626.
26. Tsuchiya S, Yamabe M, Yamaguchi Y, Kobayashi Y, Konno T, Tada K. Establishment and characterization of a human acute monocytic leukemia cell line (THP-1). Int J Cancer 1980 August; 26 (2): 171-176.
27. Raschke W C, Baird S, Ralph P, Nakoinz I. Functional macrophage cell lines transformed by Abelson leukemia virus. Cell 1978 September; 15 (1): 261-267.
28. Ralph P, Nakoinz I. Phagocytosis and cytolysis by a macrophage tumour and its cloned cell line. Nature 1975 Oct. 2; 257 (5525): 393-394.
29. Devitt A, Moffatt O D, Raykundalia C, Capra J D, Simmons D L, Gregory C D. Human CD14 mediates recognition and phagocytosis of apoptotic cells. Nature 1998 Apr. 2; 392 (6675): 505-509.
30. Devitt A, Pierce S, Oldreive C, Shingler W H, Gregory C D. CD14-dependent clearance of apoptotic cells by human macrophages: the role of phosphatidylserine. Cell Death Differ 2003 March; 10 (3): 371-382.
31. Devitt A, Gregory C D. Measurement of apoptotic cell clearance in vitro. Methods Mol Biol 2004; 282: 207-221.
32. Gregory C D, Devitt A, Moffatt O. Roles of ICAM-3 and CD14 in the recognition and phagocytosis of apoptotic cells by macrophages. Biochem Soc Trans 1998 November; 26 (4): 644-649.

1. A method for modulating the recruitment of at least one of: macrophages and monocytes (MMs), to a site of cell injury or cell death, which method comprises providing a modulator of the activity of ICAM-3 at or proximal to the site.

2. A method of treatment of a disease in a mammal in need of the same, wherein the disease is associated with undesirable infiltration of: at least one of monocytes and macrophages (MMs), to a site of cell injury or cell death, which method comprises providing an inhibitor of the activity of ICAM-3 at or proximal to the site.

3. A method as claimed in claim 2 wherein the ICAM-3 inhibitor inhibits the chemoattractant activity of ICAM-3 in respect of MMs such that administration of the ICAM-3 inhibitor inhibits the migration of MMs to the site and thereby decreases the number of MMs at the site.

4. A method as claimed in claim 2 wherein the cell death is through apoptosis.

5. A method as claimed in claim 2 wherein the disease is an inflammatory disease which is optionally an autoimmune disease, or is cancer.

6. A method as claimed in claim 5 wherein the site of cell injury or death is one wherein the presence of said MMs promotes an inflammation associated with the disease.

7. A method as claimed in claim 2 wherein the disease is selected from: atherosclerosis and the site is an atherosclerotic plaque; myocardial infarction, stroke or acute ischemia; hypertension; reperfusion injury; aortic aneurysms; vein graft hyperplasia; angiogenesis; hypercholesterolemia; congestive heart failure; Kawasaki's disease; stenosis or restenosis, particularly in patients undergoing angioplasty; rheumatoid arthritis, and glomerulonephritis; or a disease shown in Table 1 or Table 2; or a granuloma-associated disease.

8. A method as claimed in claim 2 wherein the disease is a cancer and the site is a tumor, and the method is for inhibiting at least one of: tumorigenesis and reducing cancer-associated cachexia.

9. A method as claimed in claim 2 wherein the inhibitor is provided in the proximity of the site by means of direct injection, or by use of an indwelling device coated with or containing the modulator.

10. A method as claimed in claim 9 wherein the inhibitor is provided by means of an impregnated stent.

11. A method for providing or screening for a compound having efficacy in a method, for:
modulating the recruitment of at least one of: macrophages and monocytes (MMs), to a site of cell injury or cell death,
treating a disease in a mammal in need of the same, wherein the disease is associated with undesirable infiltration of at least one of: monocytes and macrophages (MMs), to a site of cell injury or cell death, or combinations thereof;
which method comprises:
(i) providing a putative ICAM-3 inhibitor which is an ICAM-3 derived agent; and
(ii) determining its ability to inhibit chemoattraction between a chemoattractant which comprises ICAM-3 and a macrophage or monocyte.

12. A method as claimed in claim 2 wherein the ICAM-3 inhibitor:
(i) binds directly to ICAM-3 thereby reducing interaction between ICAM-3 and MMs;
(ii) binds directly to relevant ICAM-3 receptors on MMs thereby reducing interaction between ICAM-3 and MMs; or
(iii) competes with ICAM-3 for MM binding, for instance by way of being structural analogs lacking the relevant biological activity of ICAM-3.

13. A method as claimed in claim 2 wherein the ICAM-3 inhibitor is an antibody molecule.

14. A method as claimed in claim 13 wherein the antibody molecule binds an epitope in ICAM-3 at least one of: Domain 1 and Domain 2.

15. A method as claimed in claim 13 wherein the antibody molecule is a monoclonal antibody.

16. A method as claimed in claim 13 wherein the antibody molecule is a γ-immunoglobulin (IgG).

17. A method as claimed in claim 13 wherein the antibody molecule has at least the variable domain of MA4.

18. A method as claimed in claim 13 wherein the antibody molecule is monovalent.

19. A method as claimed in claim 13 wherein the antibody molecule is an antibody fragment, which is optionally an scFV antibody.

20. A method as claimed in claim 13 wherein the antibody molecule is a humanised antibody molecule.

21. A method as claimed in claim 2 wherein the ICAM-3 inhibitor is a fragment, analog or mimetic of all or part of the ICAM-3 molecule, wherein the fragment, analog or mimetic is optionally derived from the sequence of at least one of Domain 1 and Domain 2, of ICAM-3.

22. A modulator, more preferably an inhibitor, of ICAM-3 for at least one of:
modulating the recruitment of at least one of: macrophages and monocytes (MMs), to a site of cell injury or cell death, and
treating a disease in a mammal in need of the same, wherein the disease is associated with undesirable infiltration of at least one of: monocytes and macrophages (MMs), to a site of cell injury or cell death.

23. (canceled)

24. A method as claimed in claim 1 wherein the modulator is provided in the proximity of the site by means of direct injection, or by use of an indwelling device coated with or containing the modulator.

25. A method as claimed in claim 24 wherein the modulator is provided by means of an impregnated stent.
